(19) United States
(12) Patent Application Publication
(10) Pub. No.: US 2018/0126064 A1
(43) Pub. Date: May 10, 2018

(54) Storage System and Method

(71) Applicant: Aidan Jai Chandra, Atherton, CA (US)

(72) Inventor: Aidan Jai Chandra, Atherton, CA (US)

(21) Appl. No.: 15/792,545

(22) Filed: Oct. 24, 2017

Related U.S. Application Data
(60) Provisional application No. 62/412,892, filed on Oct. 26, 2016.

(51) Int. Cl. A61M 5/00; B65D 81/38

(52) U.S. Cl. CPC A61M 5/002; B65D 81/3834; A61M 2205/3673; A61M 2205/3633; A61M 2205/50; A61M 2205/18; A61M 2205/3317; A61M 2205/8206; A61M 2205/3368; A61M 2205/3306; A61M 2205/3331; A61M 2205/583; A61M 2205/581; A61M 2205/3584

(57) ABSTRACT

The present specification describes a portable system and method for storing environmentally sensitive medicines. In an embodiment, the system comprises an insulated container with an open side, where the insulated container is configured to hold one or more medicines, an insulated lid covering the open side of the insulated container, and an electronic circuit comprising a data acquisition unit, a processor in communication with the data acquisition unit, and a communication interface connected to the processor.

### CROSS-REFERENCE

The present specification relies on U.S. Patent Provisional Application No. 62/412,892, entitled “Storage System and Method”, filed on Oct. 26, 2016, for priority, which is expressly incorporated herein by reference.

### FIELD

The present specification generally relates to a protective storage system and method for storing pharmaceuticals, medicines and medical equipment.

### BACKGROUND

Physicians often advise patients to carry their medicines with them wherever they go. However, when porting the medicines to different geographies or locations, the medicine is subjected to environmental variations, such as but not limited to variations in temperature, humidity, pressure, among others. Most medicines are required to be stored within a specified temperature, light level and/or environmental range. The specified temperature ranges need to be maintained during storage. In most cases, the specified temperature and other environmental ranges may not be compatible with the environmental conditions during the user's travel. Deviations from the specified ranges may risk the effectiveness of the medicines.

Epinephrine is an auto-injecting portable device that is used by those with severe allergies. The auto-injecting device delivers emergency treatment for an anaphylactic reaction. The device is portable and carried everywhere by its users. A well-known Epinephrine auto-injecting product instructs users to store its carrier tube within a temperature range of 15° C. and 30° C. (59° F. to 86° F.). Users are typically also instructed that auto-injectors should not be exposed to extreme heat or cold and should be protected from light. In an exemplary scenario, a user travelling for outdoor sports to a geography where mid-day temperatures may soar up to 37° C. (100° F.), would compromise the efficacy of Epinephrine, resulting in possible failure during emergency situations, which are often fatal.

Thermal and refrigerating units are often used to safely store medicines that are sensitive to environmental variations. However, such units are either not portable, are fragile, too heavy or bulky to carry around. Some portable insulating units currently available for storing medicines are known to have limitations in terms of: a need for an electrical power supply for its operation; are power-consuming resulting in a low battery life; or need constant attention from the user to monitor efficacy of the insulating units.

There is a need for low-cost, light-weight, portable systems that can overcome the above limitations and provide a storage system for protection of environmentally-sensitive medications.

### SUMMARY

The following embodiments and aspects thereof are described and illustrated in conjunction with systems, tools and methods, which are meant to be exemplary and illustrative, not limiting in scope.

In some embodiments, the present specification discloses a portable pharmaceutical storage system comprising: an insulated container defining an enclosed volume in a range of no more than 1500 cubic centimeter (cm³) wherein the container comprises a base, four sides, and a lid configured to open and close to thereby provide access to the enclosed volume; a foam layer configured to fit within the enclosed volume, wherein said foam layer comprises a first groove defining a rectangular prism volume in a range of 13 cm³ to 17 cm³; and an electronic circuit positioned within the insulated container comprising: a data acquisition unit; a processor in communication with the data acquisition unit; and a communication interface connected to the processor, wherein the electronic circuit is configured to sense environmental parameters and generate an alert based upon said sensed environmental parameters.

Optionally, the electronic circuit further comprises a power source.

Optionally, the power source is a re-chargeable battery.

Optionally, the insulated container comprises an interior surface and an exterior surface that are opposite to each other, and an insulating material is attached to the interior surface.

Optionally, a material for constructing the insulated container and the lid comprise a thermoplastic polymer.

Optionally, a material for constructing the insulated container and the lid comprise at least one of stainless steel and aluminum.

Optionally, the insulated lid comprises a lock to engage with the insulated container when the insulated lid is in a closed position. Still optionally, the lock comprises at least one pair of magnets embedded on the insulated container and the insulated lid.

Optionally, the insulated lid is detachably attached to the insulated container.

Optionally, the system further comprises one or more auto-injecting pharmaceutical devices. Still optionally, the one or more auto-injecting devices comprise Epinephrine.

Optionally, the environmental parameters include one or more of temperature, light conditions, humidity, and pressure.

Optionally, the system further comprises a temperature management module configured to actively manage a temperature inside the system, and wherein the electronic circuit is configured to control the temperature management module.

Optionally, the temperature management module comprises a Peltier device Optionally, the data acquisition unit comprises one or more sensors configured to sense one or more of temperature, light conditions, humidity, and pressure.

Optionally, the communication interface comprises at least one of a visual interface, and auditory interface, and a wireless communication interface.

Optionally, the wireless communication interface is in communication with a portable electronic device.

Optionally, the visual interface is an OLED display, and wherein the display is provided on an external surface of the system.

In some embodiments, the present specification discloses a method for storing one or more auto-injecting pharmaceutical devices, the method comprising: receiving the one or more auto-injecting pharmaceutical devices in an insulated container defining an enclosed rectangular prism volume in a range no greater than 1500 cm³, wherein the insulated container comprises a top surface covered by an insulated lid; acquiring environmental data through a data acquisition unit placed within the insulated container, wherein the environmental data includes a temperature of the enclosed volume; processing the acquired environmental data by a processor in communication with the data acquisition unit; communicating the processed data through a communication interface; and activating a cooling mechanism positioned within the enclosed volume based on the acquired environmental data.

The aforementioned and other embodiments of the present invention shall be described in greater depth in the drawings and detailed description provided below.

### BRIEF DESCRIPTION OF THE DRAWINGS

These and other features and advantages of the present invention will be appreciated, as they become better understood by reference to the following detailed description when considered in connection with the accompanying drawings, wherein:

FIG. 1A illustrates a top front perspective view of a closed storage system, in accordance with some embodiments of the present specification;

FIG. 1B illustrates a side front perspective view of the storage system of FIG. 1A in an open condition, in accordance with some embodiments of the present specification;

FIG. 1C illustrates a side front perspective view of a storage system, in accordance with another embodiment of the present specification;

FIG. 1D illustrates another side front perspective view of the storage system of FIG. 1B, in accordance with some embodiments of the present specification;

FIG. 2 illustrates an exemplary embodiment of a storage system with an embedded display, in accordance with some embodiments of the present specification;

FIG. 3A illustrates a front top perspective view of another exemplary embodiment of a storage system that provides an active control over environmental conditions inside the system, in accordance with some embodiments of the present specification;

FIG. 3B illustrates a side top perspective view of the embodiment of FIG. 3A of the storage system that provides an active control over environmental conditions inside the system, in accordance with some embodiments of the present specification; and

FIG. 4 is a flow chart illustrating an exemplary method of operation of a storage system, in accordance with some embodiments of the present specification.

### DETAILED DESCRIPTION

In an embodiment, a portable protective storage system is provided to house environmentally-sensitive medicines. The system insulates and actively monitors ambience parameters of the medicine. The parameters may be communicated to a user through a visual, auditory, or other means. In an embodiment, parameters are communicated to a smartphone or any other portable electronic device through a wired or a wireless medium such as Bluetooth technology.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention. In the description and claims of the application, each of the words “comprise” “include” and “have”, and forms thereof, are not necessarily limited to members in a list with which the words may be associated.

It should be noted herein that any feature or component described in association with a specific embodiment may be used and implemented with any other embodiment unless clearly indicated otherwise.

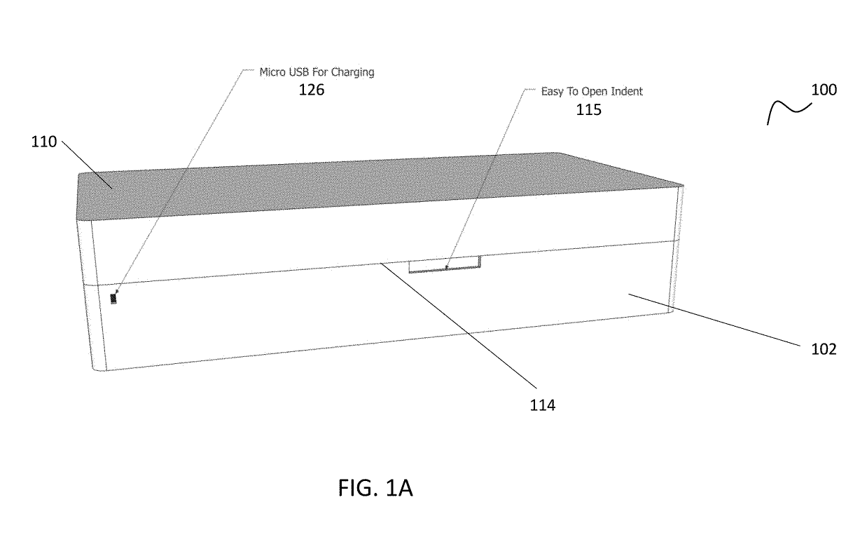

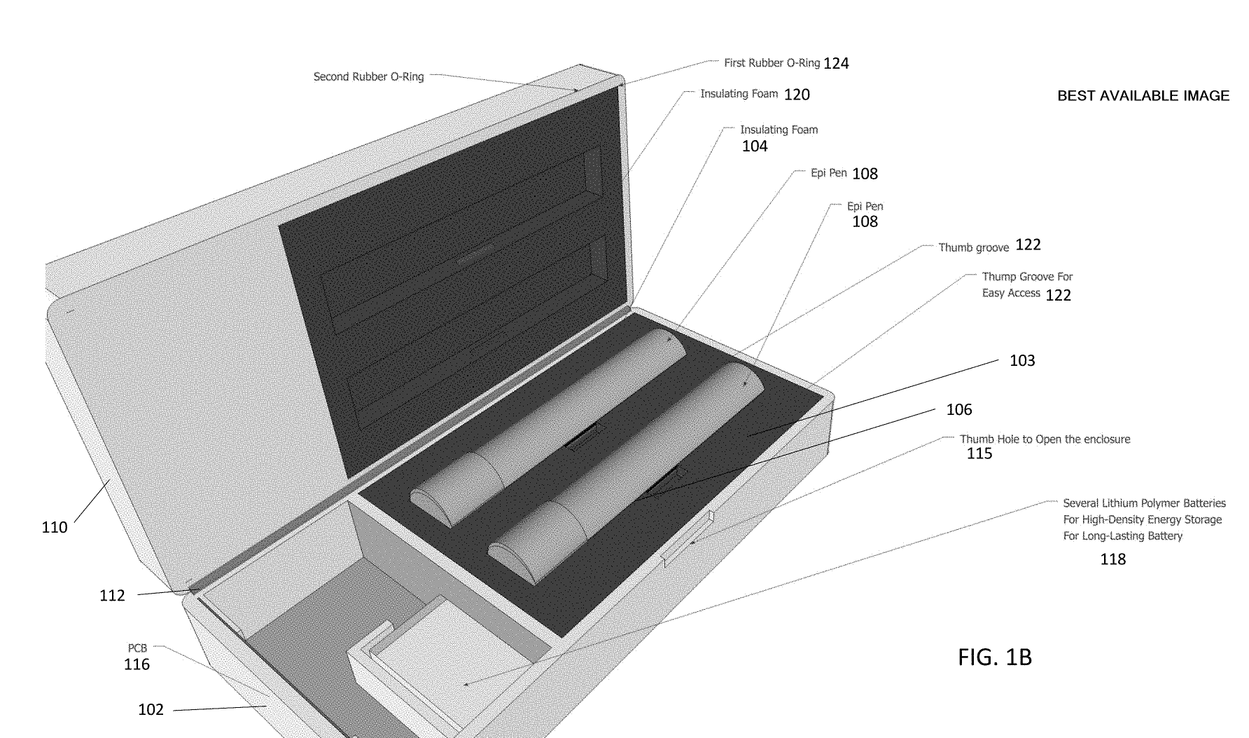

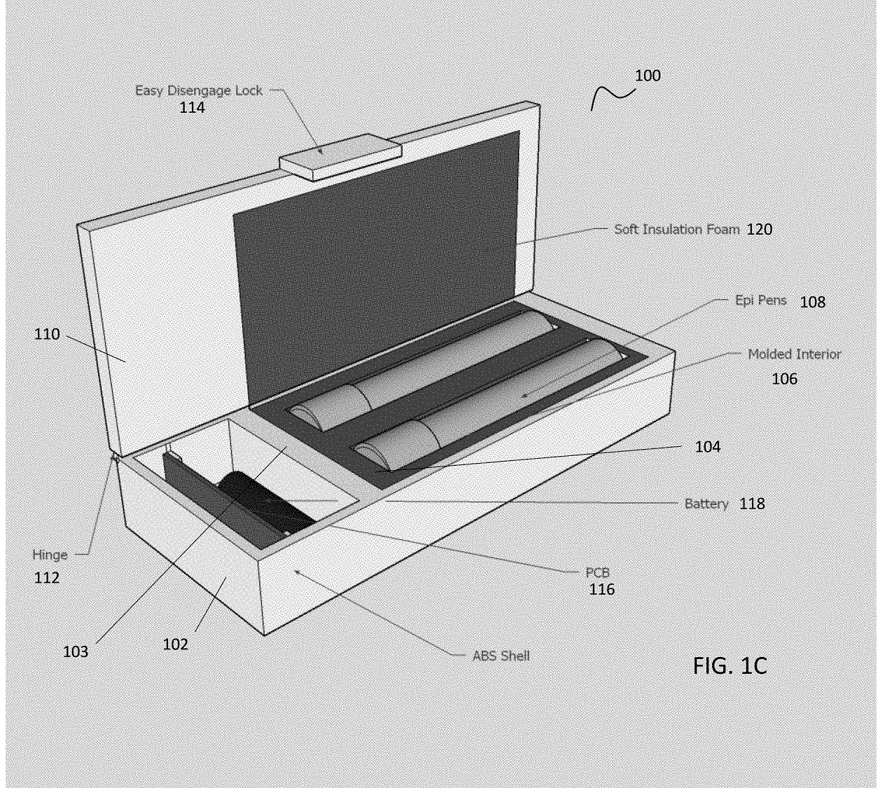

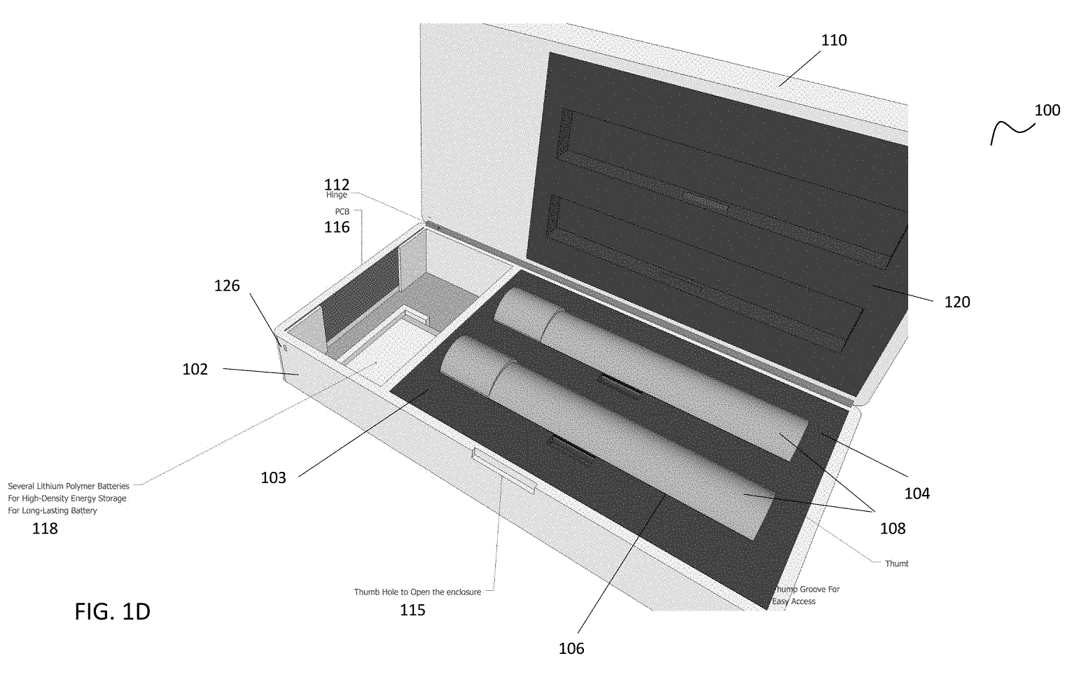

FIG. 1A illustrates a top front perspective view of a closed storage system 100, in accordance with some embodiments of the present specification. FIG. 1B illustrates a side front perspective view of open storage system 100, in accordance with some embodiments of the present specification. FIG. 1C illustrates a side front perspective view of storage system 100, in accordance with another embodiment of the present specification. FIG. 1D illustrates another side front perspective view of storage system 100, in accordance with some embodiments of the present specification.

Referring simultaneously to FIGS. 1A, 1B, 1C, and 1D, in an embodiment, storage system 100 includes an insulated container portion 102 and a lid portion 110, connected by a hinge portion 112. In embodiments, insulated container portion 102 defines an enclosed volume in a range of no more than 1500 cubic centimeters, where the container comprises a base, four sides, and lid portion 110, which is configured to open and close to provide access to the enclosed volume.

In one embodiment, insulated container portion 102 has a length ranging from 252 to 254 mm, a width ranging from 125 to 127 mm, and a height ranging from 33 to 35 mm. In embodiments, an open side 103 is also referred to as a top portion 103 of container 102.

In an embodiment, an insulated lid 110 provides a cover to open side 103 of insulated container 102. In some embodiments, the lid has a length in a range of 252 mm to 254 mm, a width in a range of 125 mm to 127 mm, and a height in a range of 23 mm to 24 mm. The insulated container portion 102, in embodiments, has an equal length and width to the lid portion 110, except that the height differs in that the lid portion 110 is shorter than the container portion 102. In embodiments, the lid portion 110 of the storage system 100 is flush with the container portion 102. Optionally, the two portions (container portion 102 and lid portion 110) are sealed with O-rings.

In embodiments, it should be noted that the dimensions provided herein for container portion 102 and lid portion 110 (and therefore, the storage system 100) are meant to be exemplary only and not limiting. Therefore, it should be understood by those of ordinary skill in the art that the storage system 100 may be of any form factor, shape and size as long as it achieves the objectives of the present invention.

In an embodiment, insulation is provided within container portion 102 in the form of a soft, insulating foam 104, which is lined along the inner surface of container portion 102. In embodiments, foam 104 has a maximum length of 176 mm, a minimum length of 172 (length range 172 mm to 176 mm), a maximum width of 118 mm, a minimum width of 114 mm (width range 114 mm to 118 mm), and a maximum height of 26 mm and a minimum height of 30 mm (height range 26 mm to 30 mm). In alternative embodiments, other known methods of insulation are incorporated with container 102. In embodiments, insulated container 102 is configured to hold one or more medicines. In some embodiments, insulating foam 104 is molded to loosely fit a medicine or a medicinal device.

FIGS. 1B, 1C, and 1D illustrate insulating foam 104 molded to receive two medicinal systems, such as auto-injecting devices 108. In one embodiment, insulating foam 104 is manufactured using polystyrene open-cell insulating foam. In embodiments, foam 104 is flexible and spongy. In embodiments, foam 104 is shaped similar to container 102, in order to fit within container 102. In one embodiment, foam 104 is sized and shaped to fit within the entirety of container 102. In another embodiment, foam 104 is sized and shaped to fit within a portion of container 102. In embodiments, foam 104 forms a soft surface within container 102. In an embodiment, and as shown in FIGS. 1B, 1C, and 1D, foam 104 comprises a substantially rectangular block of polystyrene open-cell insulating foam. Foam 104 has at least one, and preferably two parallel elongated grooves 106 molded within the rectangular block. In one embodiment, at least one groove 106 defines a rectangular prism, wherein the rectangular prism has a volume in a range of 13 cm³ to 17 cm³, to enable placement of medicinal devices. In some embodiments, grooves 106 within the rectangular block of foam 104 (forming a rectangular prism) have a length in a range of 125 mm to 140 mm, a width in a range of 10 mm to 14 mm, and a depth in a range of 4 mm to 8 mm, or corresponding to a dimension of a medicinal device 108 that system 100 is configured to store. In another embodiment, grooves 106 define a semi-cylindrical structure to enable the placement of medicinal devices such as auto-injecting devices.

In other embodiments, foam 104 has “cut-away” portions or portions that can be “pinched” away to accommodate medicines and medical devices of different dimensions. Thus, a user can simply tear away perforated or pre-notched portions of foam 104 to adjust for a snug fit of different medicines and medical devices. The container of the present specification is designed to accommodate a variety of medicines and medical devices.

In non-limiting embodiments, the present specification is designed to be used to an auto-injecting device, such as an auto-injecting device for Epinephrine (“Epi-pens”). In embodiments, insulating foam 104 is molded to enable a friction fit of an Epinephrine container or any other medicine or medicinal device 108. In some embodiments, insulating foam 104 includes thumb grooves 122 that enable the contents of container 102 to be extracted by hand easily and quickly. In embodiments, thumb grooves 122 are adjacent to grooves 106. Each groove 122 may be configured along the length of groove 106 in insulation foam 104, where groove 106 is configured to fit the auto-injecting device 108. A length of grooves 122 may be a fraction of the length of grooves 106 along which they are configured. FIGS. 1B, 1C, and 1D illustrate an exemplary groove 106 carved within foam 104, which is configured to hold two Epinephrine auto-injecting devices 108, such as Epi pens. In alternative embodiments, dimensions and design of the interior of container 102 differs with the type of medical equipment that it is used to store. While the figure illustrates a cuboid-shaped container 102, container 102 may be made from other shapes such as and not limited to a cylindrical shape. In one embodiment, container 102 is manufactured from a thermoplastic polymer, such as Acrylonitrile Butadiene Styrene (ABS). In another embodiment, container 102 is manufactured using stainless steel, aluminum, or any other material that may have the properties of impact resistance and are little affected by temperature and atmospheric humidity.

An insulating or insulation layer 120, similar to the insulating foam layer 104 within container 102 may line an inner surface of lid 110. In embodiments, soft insulating foam 120 may be manufactured using polystyrene, similar to insulating foam 104. Insulating foam 120 may cover a part of or all of the inner surface area of lid 110. In embodiments, foam 120 is structured as a rectangular block that has a length in a range of 168 mm to 172 mm, a width in a range of 83 mm to 87 mm. In an embodiment, insulation layer 120 has a thickness in a range of 4 mm to 8 mm. In an embodiment, insulation layers 104 and 120 line the inner surface area of container 102 and lid 110, respectively, where one or more medicines 108 are held.

In an embodiment, at least one set of hinged mechanism 112 connects an edge of lid 110 to a corresponding edge of container 102. Lid 110 may be detachably attached to container 102. In embodiments, while hinged mechanism 112 facilitates permanent joining of lid 110 and container 102 along one edge that is at a back side of storage system 100, a locking mechanism is configured to temporarily join, and lock, lid 110 and container 102 along another edge that is at a front side of storage system 100. In one embodiment, the locking mechanism includes one or more pairs of magnets, where each pair has one magnet on the front edge of lid 110 and the other magnet on the front edge of container 102. The magnets may be embedded in the body of system 100 such that only the surface of each magnet is aligned with the surface of front edge of lid 110 and container 102. Magnets of each pair attract each other when lid 110 and container 102 are about to be closed. Once closed, the magnets keep storage system 100 intact in the closed position (shown in FIG. 1A), till lid 110 and container 102 are forced apart by pulling both in opposite directions. In an embodiment, an indent or a groove 115 is configured partially along a central frontal edge portion of container 102. Groove 115 may enable a user to rest their thumb for ease of opening system 100. In one embodiment, four pairs of magnets are equidistantly configured along the frontal edges of lid 110 and container 102. In embodiments, the magnets are neodymium magnets. In an alternative embodiment, lid 110 is locked with the help of a locking mechanism 114 (FIG. 1C). In an embodiment, locking mechanism 114 includes an easy to dis/engage slide-lock, press-lock, or any other type of lock, so as to provide quick and reliable access to enclosed medicine 108.

In embodiments, both halves (lid 110 and container 102) of storage system 100 are lined with a sealing mechanism. The sealing mechanism enables contents of storage system 100 to be protected from the environment outside system 100, when system 100 is closed. In an embodiment, O-rings 124 are used as the sealing mechanism. O-rings 124 may be configured all around the edges of lid 110 and container 124.

In embodiments, container 102 includes an electronic circuit 116. Electronic circuit 116 may be in the form of a Printed Circuit Board (PCB). Circuit 116 may be connected to at least one data acquisition unit (not shown) that may be configured to monitor one or more types of environmental data. In embodiments, circuit 116 may be connected to data acquisition units such as, but not limited to, a humidity sensor, two or more temperature sensors, a Hall Effect sensor, a pressure sensor, and a light sensor. Sensors or data acquisition units may be placed in positions such that they are capable of measuring parameters both inside and outside of the enclosure system 100. In embodiments, temperature sensors are able to provide data on the temperature of enclosed medicine or medicinal device 108, and the temperature outside of system 100, in order to determine the potential threat to the integrity of temperature sensitive medicine/device 108 contained within system 100. In embodiments, a Hall Effect sensor is additionally connected to and communicates with a processor on circuit 116. The Hall Effect sensor detects whether storage system 100 is closed completely, or not. In the event that lid 110 is partially open for an extended period of time, the Hall Effect sensor sends a communication to the processor. The processor may be programmed to alert a user about the un-closed, open or breached state of system 100.

Additional sensors and data acquisition units are incorporated that monitor environmental data and communicate with the processor on circuit 116. Environmental data may include data pertaining to temperature, humidity, pressure, light conditions, or any other form of environmental data. A processor, such as a micro-processing unit, may be mounted on circuit 116. The processor may be in communication with the data acquisition unit. In an embodiment, the processor may also be connected to a communication interface. The communication interface may be a wired or a wireless interface. In embodiments, electronic circuit 116 is configured to sense environmental parameters through the data acquisition unit. The sensed parameters are logged and processed by the processor. When temperature, humidity, light, and pressure thresholds for the enclosed medicine are met or exceeded, the processor may generate alerts for the user of storage system 100 through a wired interface, a wireless interface, or through visual or auditory queues. In an example, if Epinephrine Auto-injectors are being stored within system 100, the system would alert the user if temperatures rise above 30 degrees Celsius or fall below 15 degrees Celsius inside of container 102. In embodiments, the alerts are generated when the monitored parameters cross pre-defined threshold for at least one parameter. In embodiments, the thresholds can be pre-defined based on the sensitivity of the medicines that are intended to be stored in system 100. Thresholds may be also programmed by a user through a software application associated with system 100. In embodiments, the application may provide a predefined list from where the user can choose using an interface on a computer or smartphone. In an embodiment, a threshold is defined as a range. Thresholds may be pre-defined for temperatures, humidity levels, pressures, and light conditions.

While an example has been provided for use of the storage system of the present specification with an Epi-pen, it should be noted by those of ordinary skill in the art, that a similar container may be used to store other sensitive medications and medical devices, such as, but not limited to insulin and glucose monitors, fertility medicines and devices, and any other medication requiring storage under environmentally stable conditions.

In an embodiment, the communication interface includes one of or a combination of: a visual interface, such as an LED; an auditory interface, such as a piezo transducer buzzer; a wireless interface, such as a Bluetooth communication interface; or any other type of a communication interface. In an embodiment, a wired or a wireless communication interface is in communication with a portable electronic device such as a smartphone. A smartphone application or any other program may be implemented on the portable electronic device to remotely process the monitored data. An exemplary smartphone companion application could include the ability to alert the user if environmental conditions are predicted to be critical for contents of system 100. An application that provides weather updates on the smartphone may be used to predict critical environmental conditions. An exemplary use of a smartphone companion application could be to alert the user if they forget system 100 with the enclosed medicine/device 108, such as by detecting a loss of bluetooth connection that was previously established and maintained between system 100 and the user's smartphone. Furthermore, the application or program may enable the user to electronically configure and track monitoring parameters within system 100. For example, the user may configure threshold temperature to be in a first range for one type of medicine, and in a second range for another type of medicine. The application or program may be further configured to present the user with different types of data to infer when and where a type of medicine may be at risk. The data may be related to different locations and geographies of the user while travelling. The application or program may include a graph of certain environmental factors in order to provide the user of system 100 with data based on location and time. The application or program may also warn the user of system 100 of weather conditions that are potentially harmful to the enclosed medicine given a location.

In embodiments, circuit 116 further includes a power source 118. Power source 118 may power the electronic components of circuit 116 and other components connected to circuit 116. In an embodiment, power source 118 is at least one light-weight battery. In an embodiment, the battery/batteries is/are re-chargeable. In some embodiments, a micro-USB 126 port is provided on the outer surface of storage system 102, such as on the outer surface of container 102. Micro-USB 126 may be used to plug in a charger to charge power source 118. The batteries used for power source 118 may be lithium-ion or lithium-polymer type batteries. The processor may also monitor the aforementioned battery and alert the user of system 100 if the battery becomes low on charge.

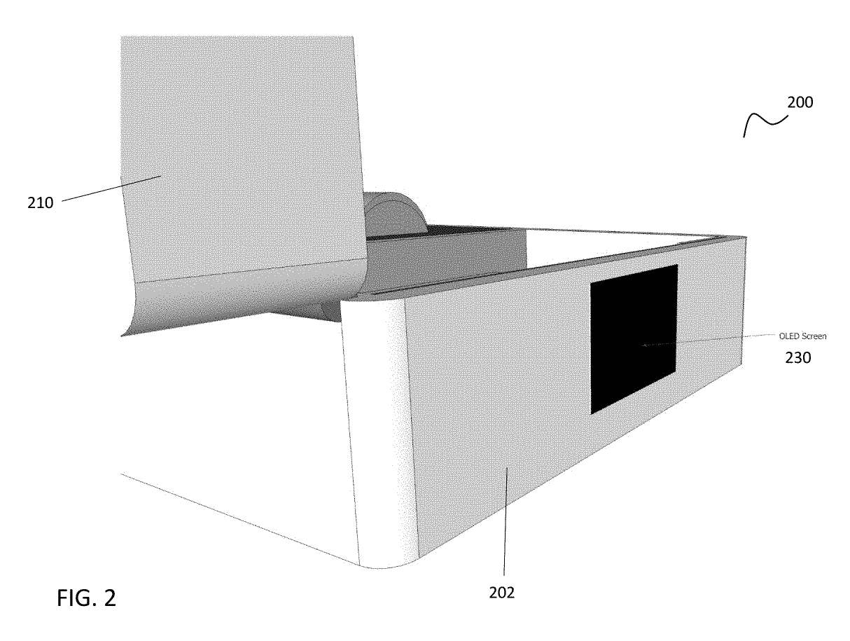

FIG. 2 illustrates an exemplary embodiment of a storage system 200 with an embedded display 230, in accordance with some embodiments of the present specification. Display 230 may be embedded on an external surface of a container 202 or a lid 210, which connect together to form storage system 200. In embodiments, various components of storage system 200 are similar to components of storage system 100 described in context of FIGS. 1A, 1B, 1C, and 1D. An additional component in the form of display 230 provides a screen to display one or more analytical information that pertains to system 200 and its contents. In an embodiment, display 230 is an OLED display. Display 230 may provide information related to one or more of a current temperature inside system 200, a current temperature outside system 200, pressure, humidity, remaining battery level, or any other information that could be useful for a user of system 200. In an embodiment, an accelerometer is embedded within system 200, which communicates with a processor provided within system 200. The accelerometer may detect and communicate to the processor, and therefore display 230, when system 200 is held by the user. Detection and communication about holding of system 200 in the user's hand(s) may, in turn, operate display 230 such that it is turned on to display information. In embodiments, when system 200 is stationary and not held by the user, display 230 is not operational.

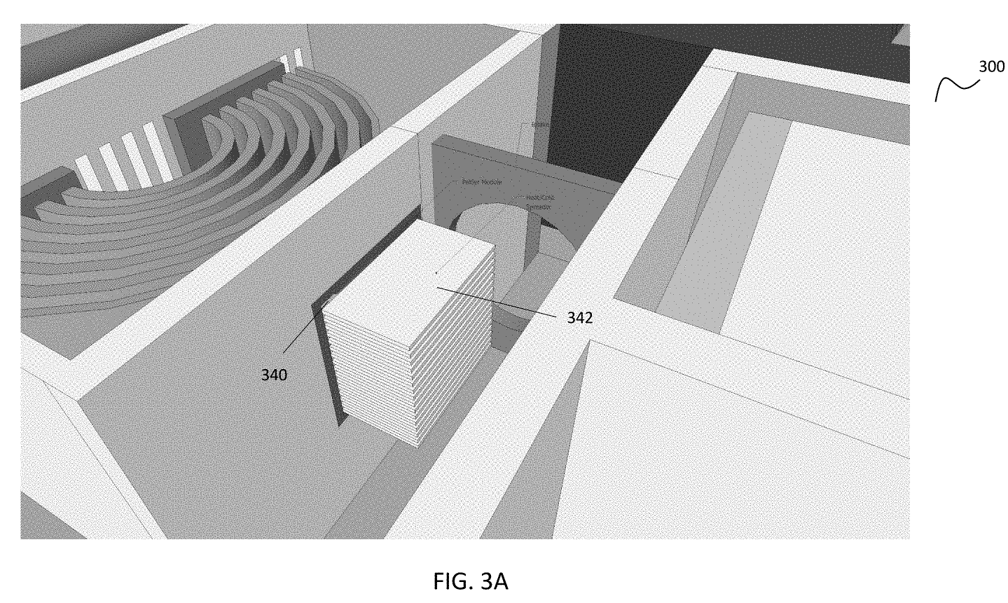

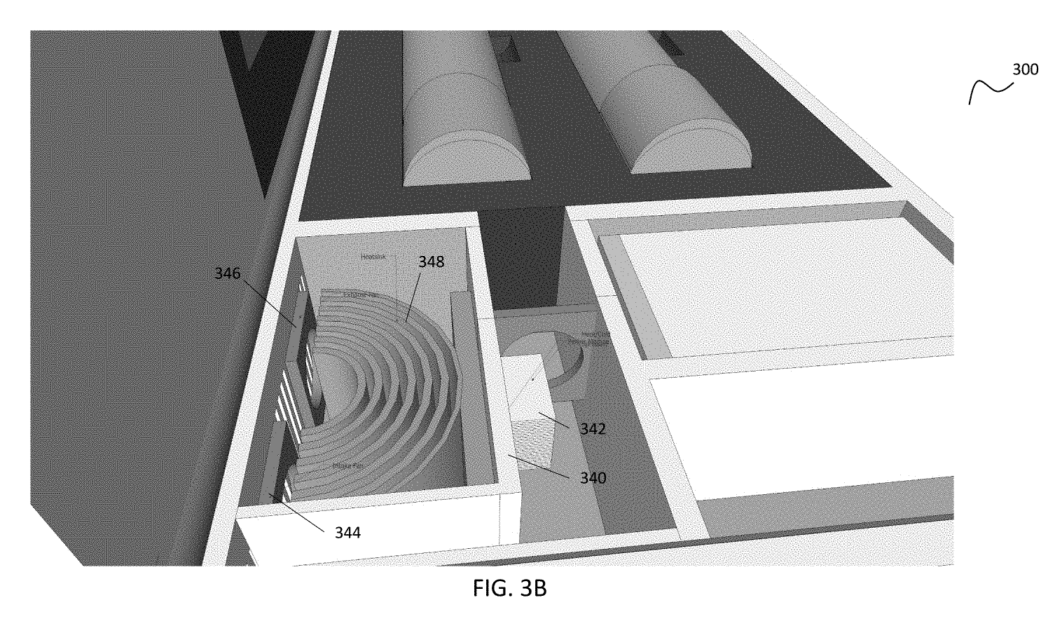

FIG. 3A illustrates a front top perspective view of another exemplary embodiment of a storage system 300 that provides an active control over environmental conditions inside system 300, in accordance with some embodiments of the present specification. FIG. 3B illustrates a side top perspective view of the embodiment of FIG. 3A of storage system 300 that provides an active control over environmental conditions inside system 300, in accordance with some embodiments of the present specification.

Referring simultaneously to FIGS. 3A and 3B, system 300 has various components that are similar to system 200. Additionally, system 300 includes components that manage at least a temperature inside (internal temperature) system 300, irrespective of a temperature outside (external temperature) system 300. In embodiments, the internal temperature is managed actively, enabling cooling of system 300 by a cooling mechanism if the external temperature is high and heating system 300 by a heating mechanism when the external temperature is low. The objective of active temperature management is to maintain the internal temperature within a range that is conducive for the medicine or medical device stored inside system 300. Active temperature management provides prolonged protection to contents of system 300 from the environmental conditions, such as temperature. In embodiments, active temperature management is provided by use of a Peltier thermoelectric module 340. Module 340 may be fitted inside system 300, where module 340 either heats or cools its ambience within system 300. In alternative embodiments, other types of temperature management devices are provided within system 300. A processor operating within system 300 may control the polarity of module 340, in order to control the temperature. The processor may trigger a need for temperature control based on temperature input provided by one or more sensors that are in communication with the processor. In an embodiment, an H-bridge control circuit is provided to control the polarity of electrical current applied to module 340. Power source of additional capacity, when compared to system 100 or system 200, may be provided to support operation of module 340. Multiple thermoelectric plates 342 may be attached to module 340, where plates 343 spread either heat or cold within system 300, based on an operation of module 340. In embodiments, system 300 also includes multiple inlets and outlets for operation of fans, including an intake fan 344 and an exhaust fan 346. In embodiments, the fans, including fans 344 and 346, operate adjacent to a heat sink 348 that is connected to module 340.

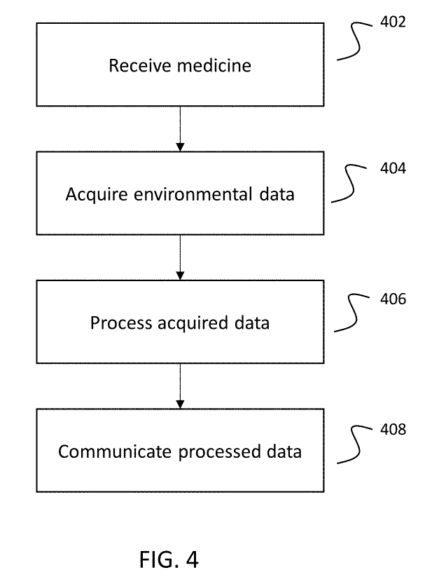

FIG. 4 is a flow chart illustrating an exemplary method of operation of storage system 100/200/300, in accordance with some embodiments of the present specification. At 402, one or more medicines may be received in insulated container 102 with an open end. In an embodiment, the open end is covered by a detachable insulated lid. In embodiments, a molded interior within container 102 is configured to hold one or more medicines such that the medicines are placed within a compact space and are discouraged from being displaced if and when a user travels with system 100/200/300. At 404, environmental data is acquired through a data acquisition unit placed within insulated container 102. In embodiments, the data pertains to one or a combination of temperature, pressure, humidity, light conditions, or any other environmental conditions that may be monitored within system 100/200/300. At 406, the acquired data is processed by a processor that is in communication with the data acquisition unit. In embodiments, processing comprises comparing the acquired environmental data to a pre-defined environmental threshold data. At 408, the processed data is communicated through a communication interface. In embodiments, the data acquisition unit, the processor, and the communication interface are connected to each other through electronic circuit 116. In embodiments, the circuit is powered by power source 118.

In an embodiment, the processed information may indicate that at least one of monitored data has crossed at least one corresponding threshold for that data. In this case, the communication interface may communicate to the user in the form of an alert. In an embodiment, the processed data is further used to actively manage environmental conditions within system 100/200/300, in order to maintain the conditions within a range that prolongs useful life of the medicines or any other content of system 100/200/300.

The above examples are merely illustrative of the many applications of the system of present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A portable pharmaceutical storage system comprising:
an insulated container defining an enclosed volume in a range of no more than 1500 cubic centimeter (cm³) wherein the container comprises a base, four sides, and a lid configured to open and close to thereby provide access to the enclosed volume;
a foam layer configured to fit within the enclosed volume, wherein said foam layer comprises a first groove defining a rectangular prism volume in a range of 13 cm³ to 17 cm³; and
an electronic circuit positioned within the insulated container comprising:
a data acquisition unit;
a processor in communication with the data acquisition unit; and
a communication interface connected to the processor, wherein the electronic circuit is configured to sense environmental parameters and generate an alert based upon said sensed environmental parameters.

2. The portable pharmaceutical storage system of claim 1, wherein the electronic circuit further comprises a power source.

3. The portable pharmaceutical storage system of claim 1, wherein the power source is a re-chargeable battery.

4. The portable pharmaceutical system of claim 1 wherein the insulated container comprises an interior surface and an exterior surface that are opposite to each other, and an insulating material is attached to the interior surface.

5. The portable pharmaceutical system of claim 1 wherein a material for constructing the insulated container and the lid comprise a thermoplastic polymer.

6. The portable pharmaceutical system of claim 1 wherein a material for constructing the insulated container and the lid comprise at least one of stainless steel and aluminum.

7. The portable pharmaceutical system of claim 1 wherein the insulated lid comprises a lock to engage with the insulated container when the insulated lid is in a closed position.

8. The portable pharmaceutical system of claim 7, wherein the lock comprises at least one pair of magnets embedded on the insulated container and the insulated lid.

9. The portable pharmaceutical system of claim 1 wherein the insulated lid is detachably attached to the insulated container.

10. The portable pharmaceutical system of claim 1 further comprising one or more auto-injecting pharmaceutical devices.

11. The portable pharmaceutical system of claim 10 wherein the one or more auto-injecting devices comprise Epinephrine.

12. The portable pharmaceutical system of claim 1 wherein the environmental parameters include one or more of temperature, light conditions, humidity, and pressure.

13. The portable pharmaceutical system of claim 12 further comprising a temperature management module configured to actively manage a temperature inside the system, and wherein the electronic circuit is configured to control the temperature management module.

14. The portable pharmaceutical system of claim 13, wherein the temperature management module comprises a Peltier device

15. The portable pharmaceutical system of claim 1 wherein the data acquisition unit comprises one or more sensors configured to sense one or more of temperature, light conditions, humidity, and pressure.

16. The portable pharmaceutical system of claim 1 wherein the communication interface comprises at least one of a visual interface, and auditory interface, and a wireless communication interface.

17. The portable pharmaceutical method of claim 16 wherein the wireless communication interface is in communication with a portable electronic device.

18. The portable pharmaceutical system of claim 16, wherein the visual interface is an OLED display, and wherein the display is provided on an external surface of the system.

19. A method for storing one or more auto-injecting pharmaceutical devices, the method comprising:
receiving the one or more auto-injecting pharmaceutical devices in an insulated container defining an enclosed rectangular prism volume in a range no greater than 1500 cm³, wherein the insulated container comprises a top surface covered by an insulated lid;
acquiring environmental data through a data acquisition unit placed within the insulated container, wherein the environmental data includes a temperature of the enclosed volume;
processing the acquired environmental data by a processor in communication with the data acquisition unit;
communicating the processed data through a communication interface; and
activating a cooling mechanism positioned within the enclosed volume based on the acquired environmental data.
